(19) United States
(12) Patent Application Publication
(10) Pub. No.: US 2008/0091250 A1
(43) Pub. Date: Apr. 17, 2008

(54) LIGHT THERAPY DESK LAMP

(75) Inventor: Steven D. Powell, Orem, UT (US)

Correspondence Address: STOEL RIVES LLP - SLC, 201 SOUTH MAIN STREET, ONE UTAH CENTER, SALT LAKE CITY, UT, 84111, US

(73) Assignee: Lumiport, LLC, Provo, UT (US)

(21) Appl. No.: 11/923,008

(22) Filed: Oct. 24, 2007

Related U.S. Application Data
(63) Continuation-in-part of application No. 10/256,866, filed on Sep. 26, 2002, now Pat. No. 7,295,608.

(51) Int. Cl. A61B 18/18

(52) U.S. Cl. 607/90

(57) ABSTRACT

A light emitting diode (LED) light therapy desk lamp device is disclosed. The light therapy desk lamp device may be used in the treatment of various conditions. The light therapy device may include multi-color LEDs for emitting a broad-spectrum non-therapeutic light and at least one narrow-spectrum therapeutic light. The light therapy device may include a removable or portable LED module control system that receives, user input including an indication of the condition to be treated. According to the user inputs, the light therapy device provides the corresponding wavelengths, intensity levels, and time interval for treatment of the condition.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings.

FIG. 1 is a is a perspective view an embodiment of a light therapy desk lamp.

FIG. 2 is a perspective view of another embodiment of a light therapy desk lamp.

FIG. 3 is an alternative view of the light therapy desk in FIG. 3.

FIG. 4A is a perspective view of an alternative embodiment of a light therapy desk lamp with a portable lamp module.

FIG. 4B is a perspective view of the portable lamp module from FIG. 4A.

FIG. 5 is a flow diagram of a control system for treating various conditions with a light therapy device.

FIG. 6 is a perspective view of an embodiment of a controller display for use with a light therapy device.

### DETAILED DESCRIPTION

Reference is now made to the figures in which like reference numerals refer to like elements. While the various aspects of the embodiments disclosed are presented in drawings, the drawings are not necessarily drawn to scale.

Those skilled in the art will recognize that the systems and methods disclosed can be practiced without one or more of the specific details, or with other methods, components, materials, etc. In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the figures herein could be arranged and designed in a wide variety of different configurations.

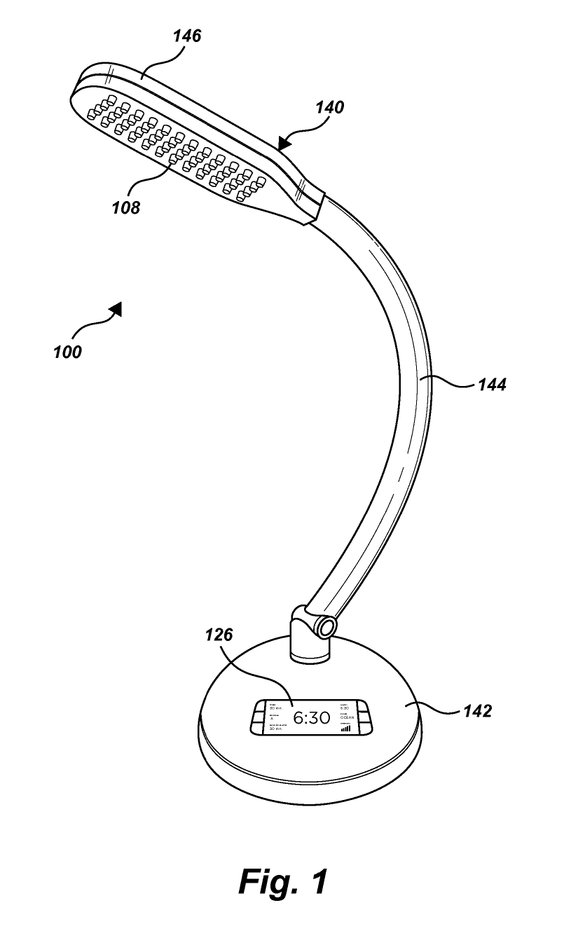

FIG. 1 represents an embodiment of a light therapy device 100 that may include a desk lamp 140. The desk lamp 140 may include a base 142, a lamp neck 144, and lamp head 146. The desk lamp 140 may also include a controller 126 which can include a display and user inputs. The desk lamp 140 may be powered by an internal or external portable power source, such as a battery. The battery power source may provide the desk lamp 140 with power such that AC power is not required. Alternatively, an AC adapter or direct AC connection may be used in other embodiments. The batteries may be stored within or proximate to the base 142.

The lamp head 146 may include one or more light emitting diodes (“LED”), such as LED array 108. The LED array 108 may produce a non-therapeutic full-spectrum (or broad-spectrum light) suitable for reading, as well as one or more narrow-spectrum therapeutic wavelengths of light. The LED array 108 may be capable of emitting multiple colors of light of different wavelengths that may be chosen to treat various conditions as desired by the user. For example, therapeutic light may be used for different types of light therapy, such as phototherapy and ocular light therapy. Phototherapy can involve treating different areas of a user's skin to resolve various skin conditions. Ocular light therapy may include projecting light into the eyes of a user for the treatment of circadian rhythm disorders, such as seasonal affective disorder (SAD), jet lag, depression, sleep disorders, shift-work disorders, fatigue, etc. More specifically, SAD is a depression that afflicts people primarily during the winter months when exposure to sunlight is often limited. Without sunlight, the brain may decrease serotonin production, often resulting in depression in some individuals.

The LED array 108 may be activated when a user depresses a button or switch disposed on the exterior of the base 142. Once activated, the LED array 108 may be configured to emit white light, narrow-band colored light in light or more wavelength ranges, or both. For example, the LED array 108 can comprise one or more bi-color (bi-polar) or multi-color (multi-polar) LEDs producing two or more discrete ranges of wavelengths as well as white light. In one configuration, the multi-color LEDs may produce a range of therapeutic wavelengths in the blue and/or red portion of the visible electromagnetic spectrum. The red wavelengths may range between 630 nanometers and 680 nanometers, while the blue wavelengths may range between approximately 400 and 500 nanometers. In one embodiment, the red wavelengths range between approximately 650 to 670 nanometers and the blue wavelengths range between approximately 405 to 420 nanometers. In another embodiment the blue wavelengths may emit light ranging from 465 to 480 nanometers. In other embodiments, the LED array 108 can include one or more tri-color LEDs producing three discrete ranges of wavelengths. Alternatively, as would understood by those having skill in the art, an LED array, such as LED array 108 may be used which can produce more than three discrete wavelengths.

In one embodiment, the LED array 108 may include one or more LEDs emitting a selected spectrum of visible light. For a non-therapeutic broad-spectrum visible light, the light from the LED array 108 can emit an effective range of 1,000 lux to 2,000 lux at a distance range of approximately 6 to 12 inches from the LED array 108.

In another embodiment, treatments for ocular light therapy may include a therapeutic spectrum of light having substantially blue colored light of wavelengths in the range of approximately 435 to 500 nanometers and generally having a distinctive, visible blue light color. For example, the LED array 108 my emit a light with a peak wavelength within the range of approximately 465 to 470 nanometers. The use of LEDs emitting such a blue-colored light have been shown to suppress melatonin. (Brainard et al., The Journal of Neuroscience, Aug. 15, 2001, 21(16):6405-6412).

In one embodiment, the LED array 108 may emit light into the eyes of a user and at distances of approximately 15 to 30 inches between the user and the LED array 108. In one example ocular light therapy may be provided from 15 to 30 minutes at distances of approximately 15 to 30 inches and at an intensity or illuminance of approximately 400 lux.

In one embodiment, the desk lamp 140 may include a controller, such as controller 126, for selecting the operating mode of the desk lamp 140. The controller 126 may be in electronic communication with the LED array 108 and may allow the user to select a broad-spectrum light for general uses such as reading. Additionally, the controller 126 may allow the user to activate desired therapeutic wavelengths of light such as red, yellow, blue, green or infrared wavelengths, or a combination thereof to treat various conditions. Additional LED wavelengths and color types may also be used. Alternatively, the controller 126 may a device for simply switching between the non-therapeutic white light and the therapeutic wavelengths of the LED array 108. The controller 126 may optionally include a display that assists a user in selecting and controlling treatment modes, timers, and other functionality features. The controller may include a memory for loading and storing multiple light treatment modes or programs for different individuals and/or different types of treatments. Treatment modes and programs may include activation of the LED array 108 to a emit a non-therapeutic broad-spectrum or a full-spectrum white light, blue wavelengths, red wavelengths, yellow wavelengths, the activation of other colors, or any combination thereof.

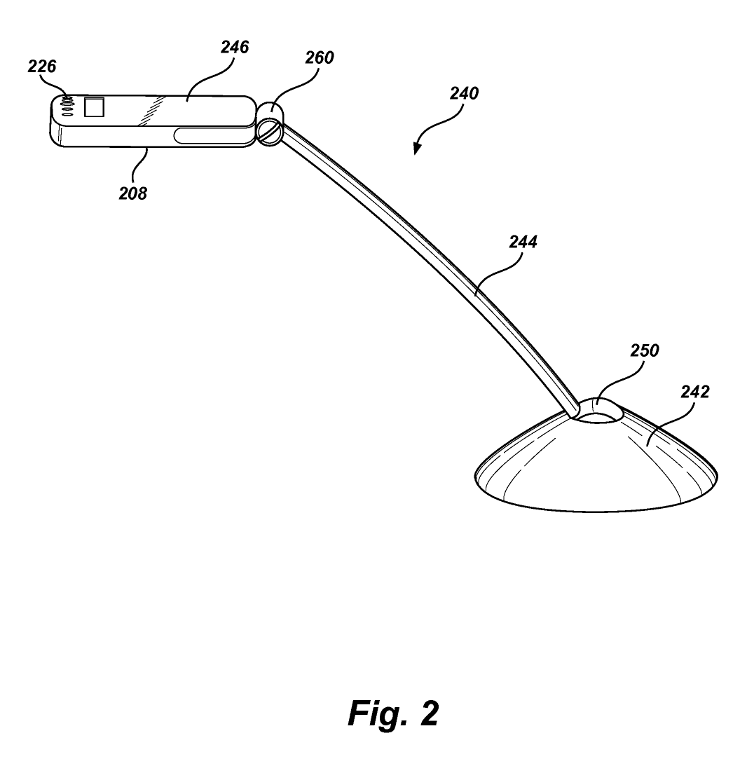

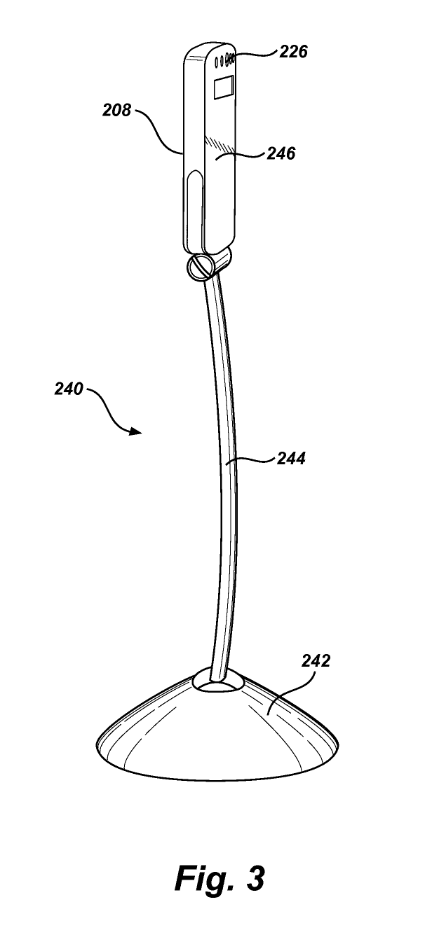

FIGS. 2 and 3 represent another embodiment of a light therapy device that may include a desk lamp 240. The desk lamp 240 may include a base 242, a lamp neck 244, and an LED module 246. The desk lamp 240 can also include base swivel 250 configured to allow the adjustment of the lamp neck 244 as shown in FIG. 3. The desk lamp 240 may also include an adjustment hinge 260 configured to allow a user to adjust the angle of the LED module 246, also shown in FIG. 3. The desk lamp 240 may also include a controller 226 which can include a display and user inputs. The desk lamp 240 may be powered by an internal or external portable power source, such as a battery. The battery power source may provide the desk lamp 240 with power such that AC power is not required. Alternatively, an AC adapter or direct AC connection may be used in other embodiments. The batteries may be stored within or proximate to the base 242 or within the LED module 246. The LED module 246 may include one or more LEDs 208, similar to LED array 108 as described previously.

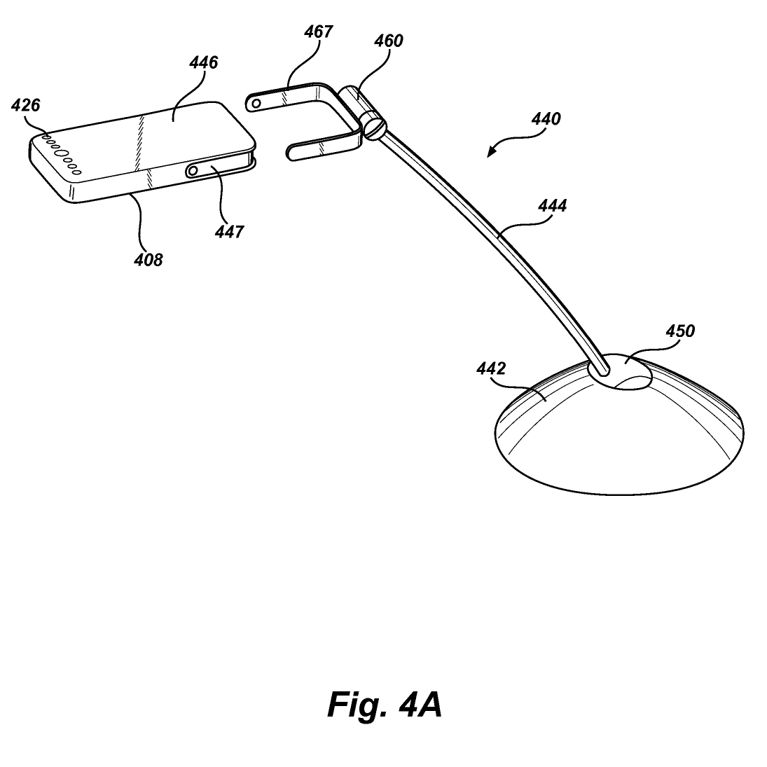

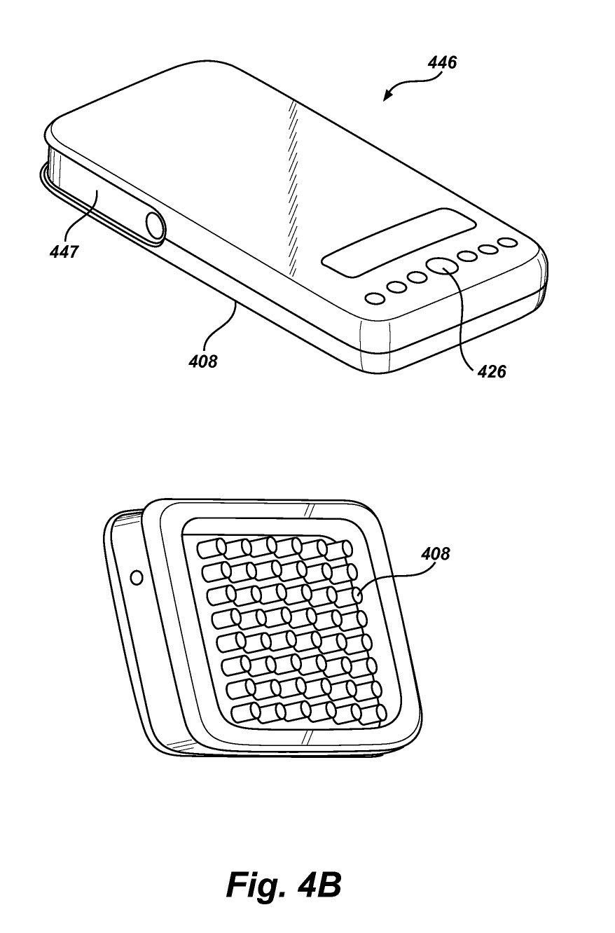

With reference to FIG. 4A, another embodiment of a light therapy device may include a desk lamp 440 with a removable and portable LED module 446. The desk lamp 440 may include a base 442 and a lamp neck 444. The lamp neck 444 can include an attachment bracket 467 configured to attach and connect the portable LED module 446 at one or more attachment points 447. The desk lamp 440 can also include base swivel 450 and an adjustment hinge 460 configured to allow a user to adjust the position of the desk lamp 444. The portable LED module 446 may also include a controller 426, similar to controller 126, which can include a display and user inputs. The portable LED module 446 may be powered by an internal or external portable power source, such as a battery. Alternatively, an AC adapter or direct AC connection may be used in other embodiments to power the LED module 446. The portable LED module 446 may include rechargeable batteries that are recharged when the portable LED module 446 is connected to the attachment bracket 467.

As shown in FIG. 4B, the LED module 446 may include one or more LEDs 408, similar to LED array 108 as described previously. The portable LED module 446 is configured to be used as a desk lamp 440 or to be used apart from the desk lamp 440 as a hand held light or treatment for SAD. The portable LED module 446 may include a fold-out support (not shown) configured to support device and direct the LEDs 408 toward a user while on a table or other flat surface. The portable LED module 446 may used while traveling, driving, or at other times when the desk lamp 444 would be too large or bulky.

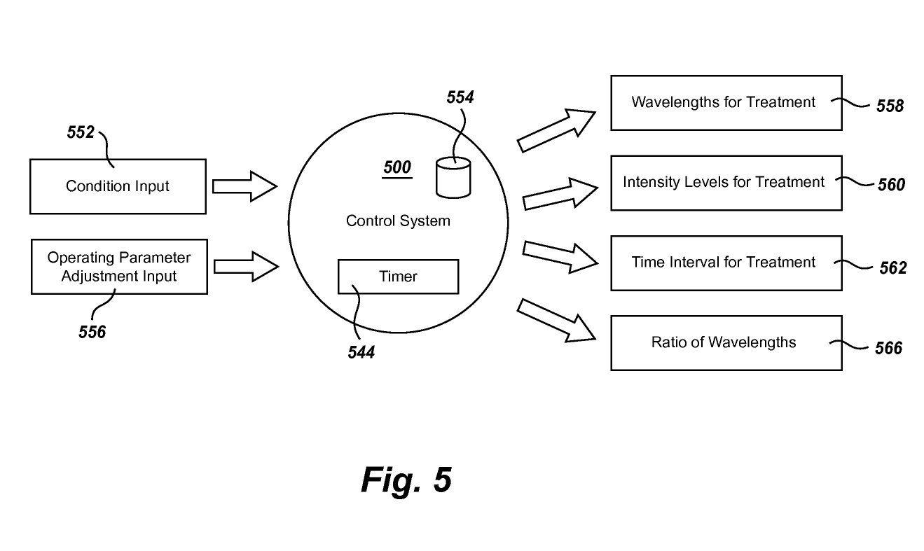

In yet another embodiment, the controller 126 or 226 or 426 may include an integrated control system, such as control system 500 shown as a block diagram in FIG. 5. The control system 500 may receive various forms of user input in order to control various treatment modes of the light therapy device. For example, a user may provide conditions or symptoms to input 552 for a circadian rhythm disorder or skin condition that may be treated by the LED array 108 of light therapy device 100. Examples of various symptoms for circadian rhythm disorders can include depression, anxiety, SAD, insomnia, fatigue, chronic sleepiness, mood swings, etc. Examples of various skin conditions may include acne, rosacea, wrinkles, inflammation, sun spots or sun damage, bacteria, blemishes, lesions or canker sores. A user may select one or more choices from a list of symptoms and/or conditions for which the control system 550 accesses operating parameters stored on a memory device 554 or database in machine readable form. The operating parameters of the light therapy device that correspond with a symptom or condition may be entered by a manufacturer or programmer of the device, or alternatively a user may provide operating parameter adjustment input 556 in accordance with a customized treatment program.

The control system 500 may access the memory device 554 containing multiple operating parameters and selects those corresponding to the condition input 552 received from the user. The light therapy device, such as the light therapy device 100, then runs according to the operating parameters corresponding with the selected condition input 552. One example of an operating parameter output of the control system 500 is a control signal corresponding to the specific wavelengths for treatment 558 of the symptom or condition selected. In other words, depending on the condition input 552 selected by the user, the control system 500 accesses the corresponding operating parameter that may indicate particular wavelengths and intensities of light to be used for treatment.

Another form of output of the control system 500 is the operating parameter that indicates the intensity levels 560 for treatment of the symptom or condition selected. For example, for a given condition input 552 inputted into the light therapy device 100 shown in FIG. 1, the intensity level output 560 of a multi-color LED may be approximately 105 mW/cm². Alternatively, for another given condition input 552, an intensity level output 560 of 92 mW/cm² may be provided by the control system 500. In another example, a user may wish to adjust the intensity level output 560 corresponding to a particular treatment. The user can adjust that particular operating parameter through input 556 indicating an increase or a decrease in intensity. Intensity adjustments may be made, for example, in percentage increments such as ±5%, ±10%, ±15%, etc.

Another operating parameter that may be controlled is the time interval for treatment 562. For example, a treatment session may last 15 minutes for a circadian rhythm disorder. However, treatment for skin conditions may be less, such as between 5 and 15 minutes, depending upon the user input. The time interval for treatment 562 may be controlled by a timer 564, which may be embodied, for example, as a Real Time Clock (RTC). Once the condition input 552 is received and the corresponding operating parameters accessed, the indicated time interval 562 can be controlled by the timer 564. Once the timer 564 reaches the time interval 562 indicated it automatically shuts off LED emission of the light therapy device.

Additionally, the operating parameters corresponding to a condition input 552 may include wavelength ratio data 566. For example, if a circadian rhythm disorder to be treated, the operating parameters might indicate that twice as much exposure to blue wavelengths as compared to red wavelengths is desired. Consequently, the wavelength ratio 566 could be 2:1, blue to red. The relative exposures of red and blue wavelengths may be determined through a quantifiable value such as light intensity or duration of exposure. Therefore, blue LED light may be emitted at twice the intensity of red LED light. Alternatively, the exposure time of blue LED light during a particular treatment interval would be twice as long as red LED light. This may be accomplished by pulsating blue LEDs twice as much as red LEDs, or by activating twice as many blue LEDs than red LEDs, or other methods known to those having skill in the art. Accordingly, a user is able to control the wavelengths emitted, the intensity levels, the time intervals for treatment, and the relative ratio of wavelengths produced by simply selecting symptoms and/or a condition.

In one example, an individual could be treated for the symptoms of SAD with ocular light therapy by emitting therapeutic wavelengths from the LED array 108 and directing them into the individual's eyes. The treatment for SAD may include placing the light therapy device 100 at approximately 20-30 inches away from the individual and exposing the individual to therapeutic wavelengths of light from approximately 440 to 480 nanometers at an intensity of approximately 1010 to 1014 photons/cm². The treatment may last from 15 to 30 minutes.

Alternatively, various skin conditions may be treated with the light therapy device as disclosed herein. For example, to treat wrinkles in the skin, blue, red and yellow wavelength bands may be used. The blue and red wavelength ranges are approximately 400 to 470 nanometers and approximately 630 to 680 nanometers, respectively. The yellow band of wavelengths may be between approximately 530 and 600 nanometers.

In treating rosacea or sun spots, a yellow range of wavelengths may be used between approximately 530 and 600 nanometers. For alternative forms of sun damage, a red band (approximately 630 to 680 nanometers) may be employed.

Blue light between approximately 400 and 470 nanometers may be used to treat and kill bacteria that may cause various forms of skin blemishes, such as acne.

Inflammation may be treated by exposing affected skin to red wavelengths (approximately 630 to 680 nanometers) and also to infrared wavelengths, which may range from about 800 nanometers to about 1000 nanometers. As discussed previously, the different wavelength ranges may be produced by an array of multi-color LEDs that can each emit a variety of colors depending on the condition to be treated.

Lesions in the skin may be treated by illuminating the affected area with red wavelengths (approximately 630 to 680 nanometers) and infrared wavelengths (approximately 800 to 1000 nanometers).

Canker sores may also be treated by irradiating the sore to red and infrared wavelengths (approximately 630 to 680 nanometers and approximately 800 to 1000 nanometers, respectively). A typical one time treatment of canker sores may have a duration of exposure between 5 and 15 minutes, with an intensity of approximately 105 mW/cm². However, multiple applications may be necessary in certain situations.

LEDs that emit a band of wavelengths in the green portion of the visible electromagnetic spectrum may also be used in treating sun spots, rosacea and wrinkles. The wavelength range associated with green light may range between approximately 100 and 530 nanometers. LED light therapy may also be used in treating dead skin and other skin problems.

The light therapy display device 100 as shown in FIG. 1, may also include a lens or diffuser (not shown) to diffuse ultra violet light or other wavelengths that may be emitted from the LED array 108. Furthermore, the LED array 108 may be removable and interchangeable with another LED array or another multi-color LED array for treatment of a different condition.

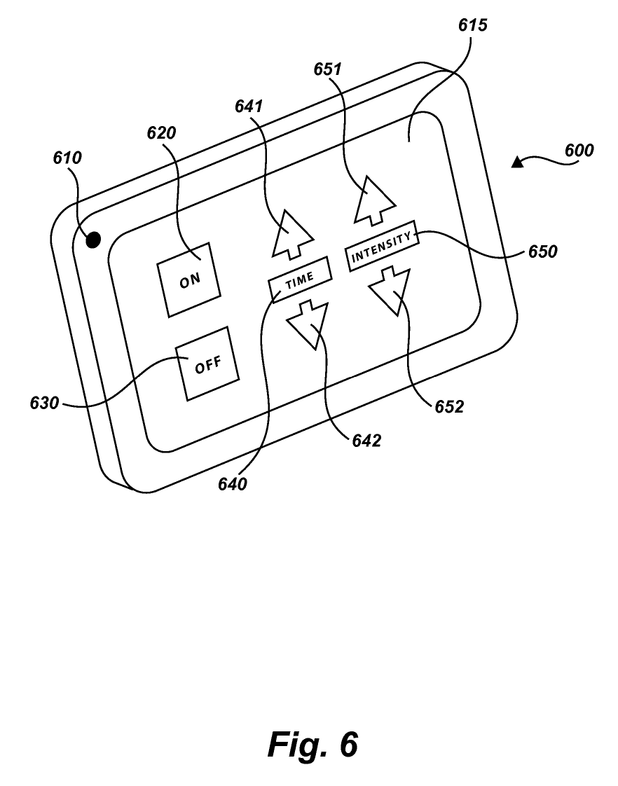

The controller 126 may be in electronic communication with a display, such as the display 600 discussed in conjunction with the description of FIG. 6. By way of example, the LCD display may show an indication of the condition selected by the user and the associated operating parameters. In some embodiments, the display may show a countdown of time left or time elapsed for the particular light therapy treatment. Furthermore, an audible alert, such as a beep, may let the user know when the treatment event has ended.

The display 600 may have a screen 615 with touch sensitive controls. The screen 615 may be a liquid crystal display (LCD) screen of the type used for touch-screen controls like those well known in the art. Alternatively, push buttons, membrane buttons, or voice activation and recognition may be used to receive user input as would be apparent to those having skill in the art.

The screen 615 may be started by activating a control button, such as control button 610 disposed on or near the display 600. When the screen 615 is active, a user may operate the light therapy display device 100 by touching the control surfaces on the screen 615. For example, the control surfaces may be operated with the user's finger or with a stylus. More particularly, a user may touch the ON control 620 to turn on a light source, like the LED array 108 shown by FIG. 1. The OFF control 630 may be used to turn off the light or terminate the light therapy and TIME controls 641 and 642 may be used to increase or decrease the duration of the treatment. The screen 615 may include other control parameters, such as INTENSITY controls 651 and 652, which may be used to control the intensity or wavelengths of the light emitted by the LED array 108. The screen 615 may also display other information and data such as the date and time, etc.

While specific embodiments and applications of light therapy display devices have been illustrated and described, it is to be understood that the invention claimed hereinafter is not limited to the precise configuration and components disclosed. Various modifications, changes, and variations apparent to those of skill in the art may be made in the arrangement, operation, and details of the devices and systems disclosed.

1. A light therapy desk lamp, comprising:
at least one LED light source configured to emit a broad-spectrum non-therapeutic light and at least one narrow-spectrum therapeutic light;
at least one controller for selecting between the non-therapeutic light and the at least one therapeutic light; and
wherein the at least one therapeutic light is selected to treat a user condition.

2. The light therapy desk lamp of claim 1, wherein the therapeutic light is a blue therapeutic light with wavelengths ranging from approximately 400 to 500 nanometers.

3. The light therapy desk lamp of claim 1, wherein the at least one LED light source is a multi-color LED capable of emitting at least two ranges of wavelengths of light from each LED.

4. The light therapy desk lamp of claim 3, wherein the multi-color LED is bi-polar and emits a range of wavelengths in a blue portion of the visible electromagnetic spectrum and a range of wavelengths in a red portion of the visible electromagnetic spectrum.

5. The light therapy desk lamp of claim 1, wherein the at least one LED light source is configured to emit the non-therapeutic light and the therapeutic light at the same time.

6. The light therapy desk lamp of claim 1, wherein the controller comprises a control system to control the LED light source according to operating parameters, the operating parameters including at least one of, wavelength selection, intensity level of light emission, and duration of light emission, such that the user selects the user condition to be treated whereupon the control system controls the light source in accordance with the operating parameters corresponding to treatment of the selected user condition.

7. The light therapy desk lamp of claim 1, wherein the controller comprises a display.

8. The light therapy desk lamp of claim 1, wherein the at least one LED light source is a portable LED module.

9. A light therapy desk lamp for the treatment of user conditions, comprising:
at least one LED light source configured to emit a broad-spectrum non-therapeutic light and at least one narrow-spectrum therapeutic light, wherein the at least one LED light source comprises a plurality of multi-color LEDs;
at least one controller for selecting between the non-therapeutic light and the at least one therapeutic light; and
a machine readable medium for storing operating parameters of the at least one LED light source, the operating parameters corresponding to treatment of at least one user condition; and
wherein the controller comprises a control system to receive input from the user indicative of a user condition to be treated, such that the control system accesses operating parameters corresponding to the indicated user condition and the control system controls the at least one LED light source in accordance with the corresponding operating parameters;
wherein the operating parameters include at least one range of therapeutic light wavelengths for treatment of each user condition.

10. The light therapy desk lamp of claim 9, wherein the at least one LED light source is configured to emit the non-therapeutic light and the therapeutic light at the same time.

11. The light therapy desk lamp of claim 9, wherein the plurality of multi-colored LEDs are each capable of emitting multiple colors of light of different wavelengths.

12. The light therapy desk lamp of claim 9, wherein the controller comprises a display configured to display the operating parameters of the at least one LED light source.

13. The light therapy desk lamp of claim 9, wherein the control system comprises a timer which is set according to the at least one time interval of the operating parameters corresponding to the indicated user condition, such that emission of the at least one LED light source is automatically discontinued when the at least one time interval has elapsed.

14. The light therapy desk lamp of claim 9, wherein the operating parameters further include at least one wavelength range ratio representing how much one range of wavelengths is emitted relative to another range of wavelengths.

15. The light therapy desk lamp of claim 9, further comprises a base and a lamp neck and a lamp head and wherein the at least one LED light source is disposed within the lamp head.

16. The light therapy desk lamp of claim 15, wherein the lamp head is a portable LED module that may be removed from the lamp neck and includes a portable power source.

17. The light therapy desk lamp of claim 15, wherein the at least one narrow-spectrum therapeutic light is in a blue portion of the visible electromagnetic spectrum ranging in wavelength from approximately 400 to 500 nanometers.

18. The light therapy desk lamp of claim 15, wherein the at least one narrow-spectrum therapeutic light is in a red portion of the visible electromagnetic spectrum ranging in wavelength from approximately 630 to 680 nanometers.

19. A method of delivering therapeutic light to an individual, the method comprising:
providing a light therapy desk lamp comprising at least one LED light source configured to emit a broad-spectrum non-therapeutic light and at least one narrow-spectrum therapeutic light, wherein the at least one LED light source comprises a plurality of multi-color LEDs;
providing at least one controller for selecting the non-therapeutic light and the at least one therapeutic light;
allowing a user to operate the controller and select between the non-therapeutic light and the at least one therapeutic light, wherein upon selection the therapeutic light, the individual indicates a treatment condition;
delivering the therapeutic light to the individual for treatment of the indicated condition.

20. The method of claim 18, wherein the indicated condition is at least one of: circadian rhythm disorders, jet lag, shift-work disorders, depression, anxiety, seasonal affective disorder, insomnia, fatigue, chronic sleepiness and mood swings.

21. The method of claim 18, wherein the indicated condition is at least one of: acne, rosacea, wrinkles, inflammation, sun damage, bacteria, blemishes and lesions.

22. The method of claim 18, wherein delivering the therapeutic light comprises delivering a therapeutic light consisting of a blue portion of the visible electromagnetic spectrum ranging in wavelength from approximately 400 to 500 nanometers.

23. The method of claim 18, wherein delivering the therapeutic light comprises delivering a therapeutic light consisting of a red portion of the visible electromagnetic spectrum ranging in wavelength from approximately 630 to 680 nanometers.

24. The method of claim 18, wherein delivering the therapeutic light comprises delivering a multi-color therapeutic light including a blue portion of the visible electromagnetic spectrum ranging in wavelength from approximately 400 to 500 nanometers and a red portion of the visible electromagnetic spectrum ranging in wavelength from approximately 630 to 680 nanometers.

25. The method of claim 18, wherein allowing a user to operate the controller comprises allowing the user to adjust at least one operating parameter of the light therapy desk lamp including adjusting at least one of: wavelength selection, intensity level of light emission, and duration of light emission.

26. The method of claim 18, wherein allowing a user to operate the controller further comprises allowing the user to select to use both the non-therapeutic light and the at least one therapeutic light at the same time.

27. The method of claim 18, wherein indicating a treatment condition comprises indicating the symptoms of the individual.
